(19) United States
(12) Patent Application Publication
(10) Pub. No.: US 2026/0087780 A1
(43) Pub. Date: Mar. 26, 2026

(54) MEDICAL DIAGNOSIS ASSISTING DEVICE, MEDICAL DIAGNOSIS ASSISTING METHOD, AND SYSTEM

(71) Applicant: CASIO COMPUTER CO., LTD., Tokyo (JP)

(72) Inventor: Mitsuyasu NAKAJIMA, Tokyo (JP)

(73) Assignee: CASIO COMPUTER CO., LTD., Tokyo (JP)

(21) Appl. No.: 19/336,763

(22) Filed: Sep. 23, 2025

(30) Foreign Application Priority Data
Sep. 24, 2024 (JP) 2024-164739

(51) Int. Cl. G06V 10/764; A61B 5/00; G06T 7/00; G06V 10/774; G06V 10/82

(52) U.S. Cl. CPC G06V 10/764; A61B 5/7267; G06T 7/0012; G06V 10/82; G06T 2207/20081; G06T 2207/20084; G06T 2207/30096; G06V 10/774; G06V 2201/03

(57) ABSTRACT

A medical diagnosis assisting device classifies a classification target in a medical image by (i) using a classifier in which cutoff values are adjusted by category in such a way that an index value that is evaluated using evaluation data divided by category comes close to a target value common to a plurality of categories and (ii) comparing an estimated value of the classification target with one of the cutoff values.

### CROSS-REFERENCE TO RELATED APPLICATIONS

This application claims the benefit of Japanese Patent Application No. 2024-164739, filed on Sep. 24, 2024, the entire disclosure of which is incorporated by reference herein.

### FIELD OF THE INVENTION

This application relates to a medical diagnosis assisting device, a medical diagnosis assisting method, and a system.

### BACKGROUND OF THE INVENTION

A technology that, using a classifier trained by machine learning, classifies a classification target in an image has been known. For example, Patent Literature 1 (Unexamined Japanese Patent Application Publication No. 2018-175226) discloses a medical image classification device that, using a determiner trained by a deep learning system, classifies medical images into a plurality of types of case areas.

### SUMMARY OF THE INVENTION

A medical diagnosis assisting device according to the present disclosure classifies a classification target in a medical image by (i) using a classifier in which cutoff values are adjusted by category in such a way that an index value that is evaluated using evaluation data divided by category comes close to a target value common to a plurality of categories and (ii) comparing an estimated value of the classification target with one of the cutoff values.

### BRIEF DESCRIPTION OF DRAWINGS

A more complete understanding of this application can be obtained when the following detailed description is considered in conjunction with the following drawings, in which:

FIG. 1 is a block diagram illustrating a configuration of a classification device according to Embodiment 1;

FIG. 2 is a diagram illustrating a configuration of a classifier according to Embodiment 1;

FIG. 3 is a diagram illustrating a relationship between sensitivity and specificity of the classifier and a cutoff value according to Embodiment 1;

FIG. 4 is a diagram illustrating sensitivities and specificities of the classifier when the cutoff value is fixed in Embodiment 1 by age group;

FIG. 5 is a diagram illustrating sensitivities and specificities of the classifier when cutoff values are set by age group in Embodiment 1 by age group;

FIG. 6 is a flowchart illustrating a flow of classifier generation processing that is executed by the classification device according to Embodiment 1;

FIG. 7 is a flowchart illustrating a flow of cutoff value adjustment processing that is executed by the classification device according to Embodiment 1;

FIG. 8 is a flowchart illustrating a flow of classification processing that is executed by the classification device according to Embodiment 1;

FIG. 9A is a diagram illustrating a result of evaluating the classifier according to Embodiment 1;

FIG. 9B is a diagram illustrating another result of evaluating the classifier according to Embodiment 1;

FIG. 9C is a diagram illustrating still another result of evaluating the classifier according

### To Embodiment 1;

FIG. 10 is a diagram illustrating a configuration of a classifier according to Embodiment 2;

FIG. 11 is a block diagram illustrating a configuration of a classifier generation device according to Embodiment 3; and

FIG. 12 is a block diagram illustrating a configuration of a classification device according to Embodiment 3.

### DETAILED DESCRIPTION OF THE INVENTION

Embodiments of the present disclosure are described below with reference to the drawings. Note that the same or corresponding parts in the drawings are designated by the same reference numerals. A classification device 100 according to Embodiment 1 is a device that classifies benignity or malignancy of a classification target in an input image, using a classifier 30 generated by machine learning. In particular, the classification device 100 according to Embodiment 1 functions as a medical diagnosis assisting device that classifies, for a medical image in which a lesion is imaged as a classification target, whether the lesion is benign or malignant. As used herein, the medical image is an image imaged for the purpose of medical diagnosis, and is an image in which a region of a living body where a disease is suspected (attention region) is imaged. A medical image is, as an example, an image obtained by imaging a skin lesion, such as a dermoscopy image. Alternatively, a medical image may be, without being limited to such an image, another type of image that can image a lesion, such as an endoscopic image, an X-ray image, a computed tomography (CT) image, and an ultrasonic image.

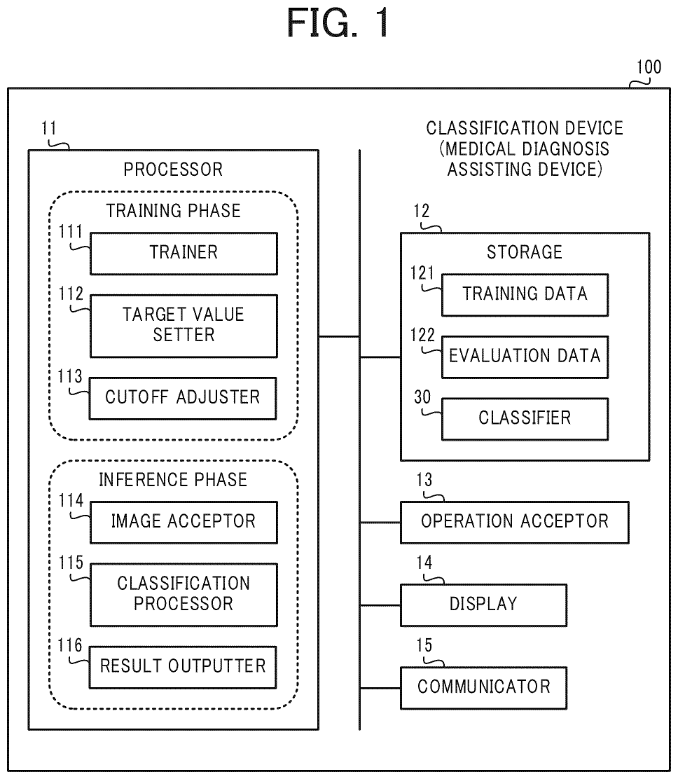

As illustrated in FIG. 1, the classification device 100 includes a processor 11, a storage 12, an operation accepter 13, a display 14, and a communicator 15. The processor 11 includes a central processing unit (CPU), a read only memory (ROM), and a random access memory (RAM). The CPU includes a microprocessor and the like, and is a central operation processor that executes various types of processing and operation. In the processor 11, the CPU retrieves a control program stored in the ROM and, using the RAM as a work memory, controls overall operation of the classification device 100. Note that processing performed by the processor 11 may be processing executed by only one CPU or processing executed by a plurality of CPUs.

The storage 12 is a nonvolatile memory, such as a flash memory and a hard disk. the storage 12 stores a program and data executed by the processor 11 as well as data generated by the processor 11. Specifically, the storage 12 stores training data 121, evaluation data 122, and the classifier 30. Details of the foregoing are described later.

The operation accepter 13 includes an input device, such as a keyboard, a mouse, and a touch panel, and accepts operation input from a user. The display 14 includes a display device, such as a liquid crystal display and an organic electro luminescence (EL) display, and displays various types of images under the control of the processor 11. The communicator 15 includes a communication interface to communicate with a device external to the classification device 100. For example, the communicator 15 communicates with an external device in conformance with a well-known communication standard, such as a local area network (LAN) and Universal Serial Bus (USB).

The processor 11 executes two phases of processing, namely a first phase that is a training phase of the classifier 30 and a second phase that is an inference phase performed by the classifier 30. The processor 11 includes, as functions in the training phase, a trainer 111, a target value setter 112, and a cutoff adjuster 113. In addition, the processor 11 includes, as functions in the inference phase, an image accepter 114, a classification processor 115, and a result outputter 116. In the processor 11, the CPU functions as the above-described functional components by retrieving programs stored in the ROM into the RAM and executing the programs to perform control. Note that in the processor 11, a single CPU may function as the functional components in the training phase and the inference phase, or a plurality of CPUs may function as the functional components in the training phase and inference phase in cooperation with one another.

First, the training phase is described. The training phase is a phase in which a classifier 30 that is capable of accurately classifying a classification target in an input image is generated using a machine learning method. As used herein, the classifier 30 is a computer program for classifying benignity or malignancy of a classification target in an input image and is a trained model trained by machine learning using the training data 121.

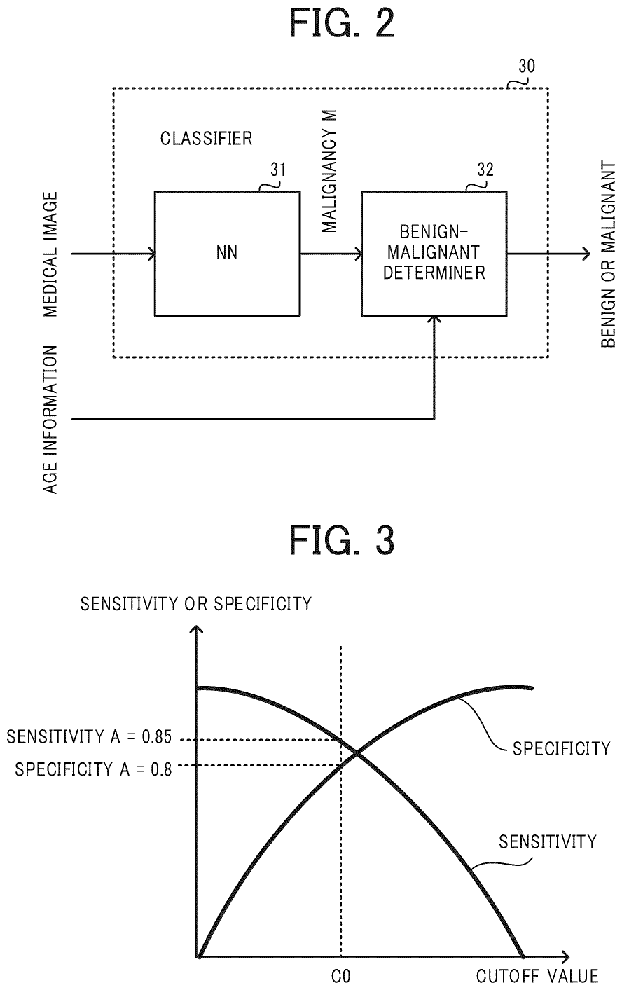

Specifically, as illustrated in FIG. 2, the classifier 30 accepts input of a medical image in which a lesion serving as a classification target is imaged and age information of a patient having the lesion imaged in the medical image. The classifier 30 outputs information indicating a result of classification of whether the lesion imaged in the medical image is benign or malignant, as output information for the input of the medical image.

More specifically, the classifier 30 includes a neural network (NN) 31 and a benign-malignant determiner 32. The NN 31 is an operation unit that executes main operation in the classifier 30. When specifically described, the NN 31 outputs malignancy M for an input medical image, using a method such as logistic regression and deep neural network (DNN). As used herein, the malignancy M is a value indicating a probability that a lesion is malignant. The malignancy M has a value of 0 or more and 1 or less and means that the closer the malignancy M is to 0, the higher the probability that the lesion is benign is and the closer the malignancy M is to 1, the higher the probability that the lesion is malignant is.

As an example, the NN 31 is constructed by a neural network with a multi-layer structure, and has a plurality of layers including an input layer into which input data is input, an intermediate layer (hidden layer) that performs an operation, such as convolution and pooling, on the input data, and an output layer (fully-connected layer) that outputs a result of the operation. The NN 31 calculates malignancy M of a lesion imaged in a medical image input to the input layer, in an intermediate layer, and outputs the calculated malignancy M from the output layer.

The benign-malignant determiner 32 outputs output information indicating whether the lesion imaged in the input medical image is benign or malignant, based on the malignancy M output from the NN 31. When specifically described, the benign-malignant determiner 32 compares the malignancy M output from the NN 31 with a preset cutoff value. The benign-malignant determiner 32 determines the lesion to be malignant when the malignancy M is greater than the cutoff value, and determines the lesion to be benign when the malignancy M is less than the cutoff value. The cutoff value is set in advance to an appropriate value between 0 and 1 in such a way that the benign-malignant determiner 32 can appropriately determine benignity and malignancy. Note that the benign-malignant determiner 32 dynamically changes the cutoff value, based on the age information input to the classifier 30, as described in detail later.

The classifier 30 outputs a determination result determined by the benign-malignant determiner 32, as output information. For example, the classifier 30 outputs value “1” as output information when the lesion is determined to be malignant, and outputs value “0” as output information when the lesion is determined to be benign. As described above, the classifier 30 classifies benignity or malignancy of a classification target in an input medical image by comparison between a predicted value of the benignity or malignancy and a cutoff value.

Returning to FIG. 1, in the training phase, the trainer 111 performs machine learning using the training data 121. The training data 121 are a data set (data set for training) used by the trainer 111 to execute the machine learning. The training data 121 include a plurality of input images for training (hereinafter, referred to as “training image”) used as teacher data. Each of the plurality of training images is an image in which a lesion is imaged, and is an image where a correct answer to whether the imaged lesion is benign or malignant is known in advance. In the training data 121, to each training image, a correct malignancy LM that indicates a correct answer with respect to a lesion imaged in the training image is attached as a teacher label (correct label) in advance. The correct malignancy LM is represented by a binary value of 1 or 0, and has 1 when the lesion is malignant and 0 when the lesion is benign. As the correct malignancy LM, for example, a result of a pathological diagnosis (biopsy) can be used.

The trainer 111 performs machine learning, using the plurality of training images included in the training data 121 as teacher data. When specifically described, the trainer 111 inputs each of the plurality of training images included in the training data 121 to the classifier 30. In the classifier 30, the NN 31 calculates and outputs an estimated value of the malignancy M of a lesion imaged in the input training image. The trainer 111 adjusts calculation parameters of the NN 31, using an error back-propagation method or the like in such a way that the malignancy M output from the NN 31 comes close to correct malignancies LM attached to the input training images. The calculation parameters of the NN 31 are, for example, weights of connections between layers in the neural network in the NN 31, in other words, weights indicating connection strengths between a plurality of neurons (nodes). By adjusting the calculation parameters, estimated values of the malignancy M that the NN 31 outputs for input training images change. The trainer 111 adjusts the calculation parameters by changing the calculation parameters in various manners in such a way that the malignancy M output from the NN 31 comes close to the correct malignancy LM to the extent possible. The trainer 111 optimizes the calculation parameters of the NN 31 by executing such adjustment processing of the calculation parameters for each of the plurality of training images included in the training data 121, and thereby constructs the neural network in the NN 31.

Returning to FIG. 1, the target value setter 112 sets a target value for a classification index that is an index value relating to classification performed by the classifier 30. As used herein, the classification index is an index value for evaluating performance of the classification of benignity or malignancy performed by the classifier 30. Specifically, the target value setter 112 sets a target value for sensitivity of the classifier 30 and a target value for specificity of the classifier 30 as target values of classification indices. The sensitivity is a ratio of cases where a malignant case is correctly classified as malignant. In contrast, the specificity is a ratio of cases where a benign case is correctly classified as benign. The sensitivity and the specificity have a trade-off relationship, and when the sensitivity is set too high, the specificity becomes low, and conversely, when the specificity is set too high, the sensitivity becomes low.

When described in more detail, as illustrated in FIG. 3, the sensitivity and specificity of the classifier 30 vary depending on the cutoff value used in the benign-malignant determiner 32. For example, when the cutoff value is set too high, a classification result is biased toward benignity, as a result of which the specificity becomes higher and the sensitivity becomes lower. In contrast, when the cutoff value is set too low, a classification result is biased toward malignancy, as a result of which the sensitivity becomes higher and the specificity becomes lower. Therefore, by changing the cutoff value used in the benign-malignant determiner 32, a combination of sensitivity and specificity, in other words, balance between sensitivity and specificity, can be adjusted. Note that since FIG. 3 illustrates a relationship among the cutoff value, the sensitivity, and the specificity in a simplified manner, the illustrated relationship is not necessarily accurate.

Meanwhile, a classification index, such as sensitivity and specificity, varies depending on a difference in a category, including age, between classification targets. For example, the sensitivity or specificity in classification of a lesion of a patient in a lower age group is not necessarily the same as the sensitivity or specificity in classification of a lesion of a patient in a higher age group. In particular, when there is a bias among categories in a plurality of training images included in the training data 121, a situation in which the sensitivity or specificity in a specific category is largely different from other categories may occur. To avoid such a situation and generate a classifier 30 capable of performing stable classification across any category, it is desirable to stabilize the balance between sensitivity and specificity to the extent possible, regardless of category. The target value setter 112 sets target values for sensitivity and specificity for that purpose. The following description is made using age groups as an example of the category.

First, the target value setter 112 sets a target value common to a plurality of categories (a plurality of age groups) as a target value for each of sensitivity and specificity. For that purpose, the target value setter 112 uses the evaluation data 122. As used herein, the evaluation data 122 are a data set used to evaluate a classification index of the classifier 30. The evaluation data 122 include a plurality of input images for evaluation (hereinafter, referred to as “evaluation images”). To each of the plurality of evaluation images, a correct malignancy LM of an imaged lesion is attached as a teacher label, as with the training images. In addition to the teacher label, to each of the plurality of evaluation images, information about an age LA of a patient having an imaged lesion is attached. Note that all or some of the plurality of evaluation images in the evaluation data 122 may be the same as the training images. In addition, the machine learning in the NN 31 and adjustment of the cutoff value in the benign-malignant determiner 32 may be performed using only the training data 121 by a cross validation method.

The target value setter 112 inputs each of the plurality of evaluation images included in the evaluation data 122 to the classifier 30 without dividing the plurality of evaluation images by age group (by category). In other words, although to each of the evaluation images included in the evaluation data 122, information about the age LA of a patient having an imaged lesion is attached, the target value setter 112, without using the information about the age LA, inputs the evaluation images imaging lesions from all age groups to the classifier 30.

When the evaluation images are input to the classifier 30, the target value setter 112 compares output information indicating benignity or malignancy that is output from the classifier 30 for input of each evaluation image with the correct malignancy LM of the evaluation image. The target value setter 112 determines whether or not the output information and the correct malignancy LM coincide with each other for each evaluation image, and calculates sensitivity and specificity, which serve as classification indices. Specifically, the target value setter 112 acquires a value of the “sensitivity” by calculating a ratio of the number of pieces of output information from the classifier 30 indicating malignancy (1) to the number of inputs of a plurality of evaluation images the correct malignancies LM of which indicate malignancy (1). In addition, the target value setter 112 acquires a value of the “specificity” by calculating a ratio of the number of pieces of output information from the classifier 30 indicating benignity (0) to the number of inputs of a plurality of evaluation images the correct malignancies LM of which indicate benignity (0).

The target value setter 112 obtains a plurality of combinations of sensitivity and specificity by executing such calculation of sensitivity and specificity by changing the cutoff value to a plurality of values. The target value setter 112 sets sensitivity and specificity in a combination that satisfies a specific condition among the obtained plurality of combinations as a sensitivity A and a specificity A, which serve as target values, respectively. As used herein, the specific condition is a condition that does not excessively reduce both the sensitivity and the specificity and that allows high values to some extent to be achieved for both the sensitivity and the specificity. For example, a condition requiring that both the sensitivity and the specificity are greater than or equal to predetermined lower limits can be set as the specific condition. The lower limit can be set to an appropriate value, such as 0.7 and 0.75.

More specifically, in addition to the condition requiring that both the sensitivity and the specificity are greater than or equal to the predetermined lower limits, a condition requiring that the sensitivity is higher than the specificity is set as the specific condition in order to minimize the number of cases of missing a malignant case. In other words, the target value setter 112 sets the target value for the sensitivity higher than the target value for the specificity. In the example of FIG. 3, when the cutoff value is set to C0, the sensitivity becomes 0.85 and the specificity becomes 0.8. The sensitivity (=0.85) and the specificity (=0.8) as described above satisfy both the condition requiring that the sensitivity and the specificity are higher than the lower limits and the condition requiring that the sensitivity is higher than the specificity. Therefore, the target value setter 112 can set the sensitivity (=0.85) and the specificity (=0.8) at the cutoff value C0 as the target values for the sensitivity and the specificity, respectively.

Note that although it is conceivable that there typically exists a plurality of combinations of sensitivity and specificity that satisfies the specific condition, the target value setter 112 may automatically set a combination of sensitivity and specificity that serves as the target values from among such a plurality of combinations. Alternatively, the user may manually select a desired combination serving as the target values from a plurality of combinations. The following description is made using as an example a case where, as illustrated in FIG. 3, the target value setter 112 sets the sensitivity (=0.85) and the specificity (=0.8) respectively as target values of sensitivity and specificity common to a plurality of age groups (hereinafter, referred to as “sensitivity A” and “specificity A,” respectively). On this occasion, the target value setter 112 sets the cutoff value C0, which enables the sensitivity A and the specificity A to be achieved, as a cutoff value common to a plurality of age groups.

Returning to FIG. 1, the cutoff adjuster 113 sets cutoff values by age group. In other words, the cutoff adjuster 113 individually sets a cutoff value for each of a plurality of age groups, in contrast to the target value setter 112 setting a cutoff value C0 common to the plurality of age groups. When specifically described, in the classification using the cutoff value C0 set by the target value setter 112, the sensitivity A and the specificity A can be achieved for the entire evaluation data 122. Meanwhile, when the plurality of evaluation images included in the evaluation data 122 is divided by age group, there are some cases where the sensitivity A and the specificity A cannot be achieved with the cutoff value C0, depending on age group.

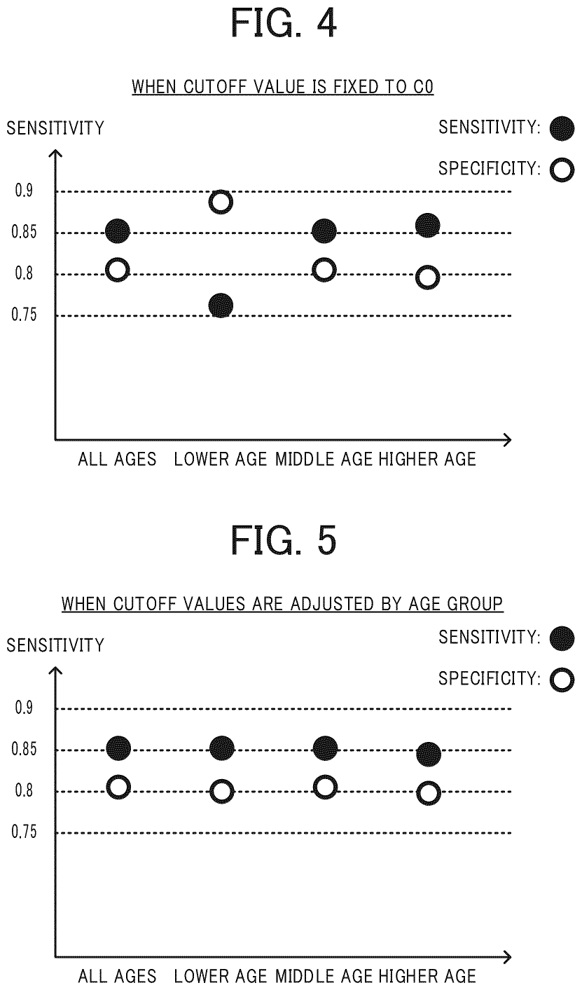

For example, in FIG. 4, the sensitivity and specificity when the cutoff value, which is used by the benign-malignant determiner 32, is fixed to C0 are illustrated with respect to each of all age groups, a lower age group, a middle age group, and a higher age group. In this example, the lower age group, the middle age group, and the higher age group represent, as an example, ranges of 0 to 39 years old, 40 to 59 years old, and 60 years old or older, respectively. When described in more detail, first, the plurality of evaluation images included in the evaluation data 122 are divided into three age groups, namely the lower age group, the middle age group, and the higher age group, according to the age of a patient imaged in each evaluation image. Next, results each of which is acquired by evaluating the sensitivity and specificity of the classifier 30 with respect to one of the three age groups, using evaluation images divided into respective age groups are indicated as data for the lower age group, the middle age group, and the higher age group in FIG. 4. In contrast, the data for all age groups in FIG. 4 are a result of evaluating the sensitivity and specificity of the classifier 30, using all of the plurality of evaluation images included in the evaluation data 122 without dividing the evaluation images by age group.

In FIG. 4, the sensitivity and specificity in the all age groups are the sensitivity A (=0.85) and the specificity A (=0.8), which serve as the target values set by target value setter 112, respectively. In addition, the sensitivity and specificity in the middle age group and the higher age group do not largely differ from the sensitivity and specificity in the all age groups and are approximately 0.85 and 0.8, respectively. In contrast, the sensitivity and specificity in the lower age group are largely different from the sensitivity and specificity in other age groups, and the sensitivity is lower than the specificity. Such a situation may arise when the probability of occurrence of malignant cases in the lower age group is low and the number of samples of training images from the lower age group in the training data 121 is small. To avoid such imbalance between the sensitivity and the specificity in one of the age groups and ensure the classification to be performed in a stable manner across all age groups, the cutoff adjuster 113 individually sets cutoff values by age group.

When specifically described, the cutoff adjuster 113 first divides the plurality of evaluation images included in the evaluation data 122 by age group. Specifically, as with FIG. 4, the cutoff adjuster 113 divides the plurality of evaluation images into three age groups, namely the lower age group (0 to 39 years old), the middle age group (40 to 59 years old), and the higher age group (60 years old or older), according to the age of a patient imaged in each evaluation image. Note that a method of division into age groups is not limited to this example. In addition, the number of age groups into which the evaluation images are divided is not limited to three, and may be two or four or more.

Next, the cutoff adjuster 113 adjusts the cutoff value for each age group in such a way that the classification indices of the classifier 30 when evaluation is performed using the evaluation data 122 divided by age group comes close to the target values set by the target value setter 112. When specifically described, the cutoff adjuster 113 calculates a loss function E expressed by the following equation (1) with respect to each piece of evaluation data 122 divided by age group by variously changing the cutoff value used in the benign-malignant determiner 32. The loss function E is a function for evaluating to what extent the sensitivity and specificity of the classifier 30 have come close to the sensitivity A and the specificity A, which serve as the target values for the sensitivity and the specificity, respectively.

E=α×((sensitivity A)−(sensitivity))²+(1−α)×((specificity A)−(specificity))² (1)

In the above-described equation (1), α is a parameter that indicates weights of the sensitivity term and the specificity terms in the loss function E. The parameter α is an external configuration variable that the user can freely set within a range of 0 or more and 1 or less. For example, when the sensitivity and the specificity are treated equally, the parameter α is set to 0.5. Alternatively, when the cutoff value is adjusted with a priority to the sensitivity over the specificity, the parameter α may be set to a value greater than 0.5.

The cutoff adjuster 113 inputs evaluation images to the classifier 30 with respect to each division of the evaluation data 122 divided by age group and determines whether or not output information indicating benignity or malignancy, which is output from the classifier 30 for input of each evaluation image, coincides with the correct malignancy LM of the evaluation image. The cutoff adjuster 113 evaluates the sensitivity and the specificity based on the determination results, and calculates a loss function E from the evaluated sensitivity and specificity. The cutoff adjuster 113 repeats such processing of calculating a loss function E by changing the cutoff value to a plurality of values and thereby searches for a cutoff value that causes the loss function E to have a minimum value, that is, come closest to zero. As a result of the search, the cutoff adjuster 113 determines the cutoff value that minimizes the loss function E as a cutoff value for the age group.

The cutoff adjuster 113 executes such processing of searching for a cutoff value for each division of the evaluation data 122 that are divided into evaluation data for the lower age group, evaluation data for the middle age group, and evaluation data for the higher age group, and thereby individually determines a cutoff value for each of the lower age group, the middle age group, and the higher age group. Because of this configuration, the cutoff adjuster 113 independently determines a cutoff value C1 for the lower age group, a cutoff value C2 for the middle age group, and a cutoff value C3 for the higher age group. As used herein, each of the cutoff values C1 to C3 for respective age groups means a cutoff value used by the benign-malignant determiner 32 when medical images in which lesions of patients belonging to a corresponding age group are imaged are input to the classifier 30. Note that the cutoff values C1to C3 for the respective age groups may become the same value as the cutoff value C0 common to all the age groups as a result of adjustment.

In FIG. 5, an example of the sensitivity and specificity of the classifier 30 with respect to the evaluation images for each of the all age groups, the lower age group, the middle age group, and the higher age group in a case where cutoff values individually adjusted by age group by the cutoff adjuster 113 are used is illustrated. It can be confirmed that the sensitivity and specificity in the lower age group in FIG. 5 have come close to the sensitivity A (=0.85) and the specificity A (=0.8), which serve as the target values that are set by the target value setter 112, compared with the case in FIG. 4. Note that since the sensitivity and specificity in the middle age group and the sensitivity and specificity in the higher age group are close to the sensitivity and specificity in the all age groups even in the case in FIG. 4, there is no significant change in FIG. 5. Since as described above, the cutoff adjuster 113 individually adjusts the cutoff values by age group, it is possible to prevent balance between the sensitivity and specificity of the classifier 30 from being disrupted in a specific age group. Therefore, it becomes possible to generate a classifier 30 that can perform classification in a stable manner across any age group.

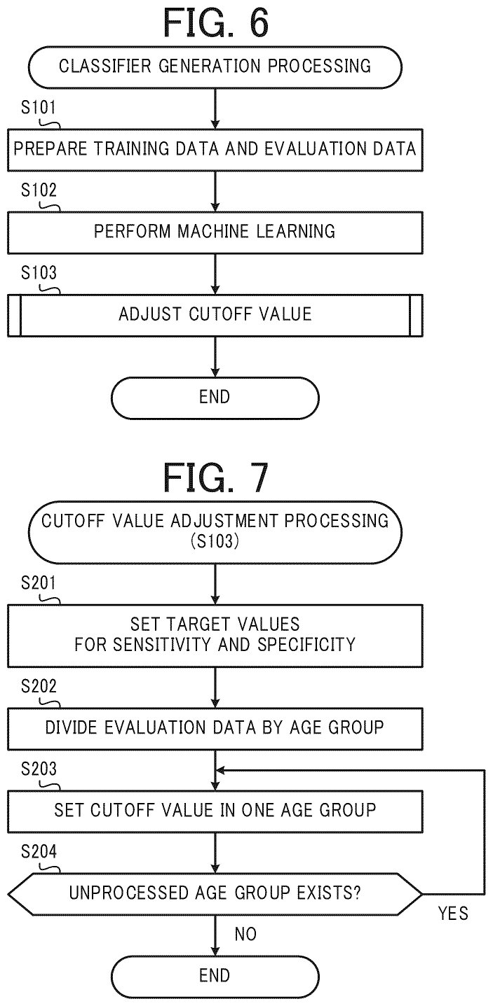

Next, with reference to FIG. 6, a flow of classifier generation processing executed by the classification device 100 in the training phase is described. The classifier generation processing illustrated in FIG. 6 is started when the operation accepter 13 accepts a start instruction from the user. The classifier generation processing illustrated in FIG. 6 is an example of a classifier generation method.

When the classifier generation processing is started, the processor 11 prepares the training data 121 and the evaluation data 122 (step S101). Next, the processor 11 functions as the trainer 111 and performs machine learning, using the training data 121 (step S102). When specifically described, the processor 11 inputs each training image included in the training data 121 to the classifier 30. The processor 11 adjusts calculation parameters of the NN 31 in such a way that malignancy M output from the classifier 30 comes close to correct malignancies LM attached to the input training image. When performing machine learning, the processor 11 adjusts cutoff values used for determination of benignity or malignancy in the benign-malignant determiner 32 (step S103). Details of cutoff value adjustment processing in step S103 are described with reference to FIG. 7.

When the cutoff value adjustment processing illustrated in FIG. 7 is started, the processor 11 functions as the target value setter 112 and sets target values that are target values for sensitivity and specificity serving as classification indices of the classifier 30 and that are common to a plurality of age groups (step S201). When specifically described, the processor 11 evaluates the sensitivity and specificity of the classifier 30, using the evaluation data 122 that are not divided by age group. The processor 11 repeats such evaluation of the sensitivity and specificity by changing the cutoff value to a plurality of values, and sets, among a plurality of combinations of sensitivity and specificity obtained through the evaluation, a combination of sensitivity and specificity that satisfies a specific condition, as the target values. The processor 11 sets a sensitivity A and a specificity A at a cutoff value C0, as illustrated in, for example, FIG. 3.

When the target values are set, the processor 11 divides the evaluation data 122 by age group (step S202). When specifically described, the processor 11 divides, based on information about age LA that is attached to each of the evaluation images included in the evaluation data 122, the evaluation images into groups of evaluation images corresponding to a plurality of age groups, such as the lower age group, the middle age group, and the higher age group. When the evaluation data 122 are divided, the processor 11 functions as the cutoff adjuster 113 and, using the evaluation data 122 divided into groups of evaluation images corresponding to the plurality of age groups, sets a cutoff value for one age group of the plurality of age groups (step S203). When specifically described, the processor 11 selects one age group out of the plurality of age groups. The processor 11 evaluates the sensitivity and specificity of the classifier 30, using evaluation data 122 of the selected age group, and calculates a loss function E expressed by the above-described equation (1). The processor 11 executes such calculation of a loss function E for each of a plurality of cutoff values, and sets a cutoff value that minimizes the loss function E as a cutoff value for the selected age group.

When having adjusted the cutoff value for the one age group, the processor 11 determine whether or not there exists an unprocessed age group within the plurality of age groups (step S204). When an unprocessed age group exists (step S204; YES), the processor 11 returns the process to step S203. The processor 11 newly selects an unprocessed age group and sets a cutoff value for the newly selected age group. In this way, the processor 11 individually sets a cutoff value for each of the plurality of age groups. When finally there exists no unprocessed age group (step S204; NO), the processor 11 terminates the cutoff value adjustment processing. Consequently, the classifier generation processing illustrated in FIGS. 6 and 7 terminates.

Returning to FIG. 1, second, the inference phase is described. The inference phase is a phase in which, using the classifier 30 that is generated in the training phase, whether an unknown lesion in an unknown medical image is benign or malignant is classified. In the inference phase, the image accepter 114 accepts input of an unknown medical image that serves as a classification target. As used herein, the unknown medical image is an image in which a lesion for which whether the legion is benign or malignant is unknown is imaged. The image accepter 114 accepts, in accordance with an instruction from the user accepted through the operation accepter 13, a specification of an unknown medical image serving as a classification target from a plurality of medical images stored in the storage 12 in advance. Alternatively, the image accepter 114 may accept an unknown medical image serving as a classification target from the outside by the communicator 15.

In addition, the image accepter 114 accepts input of age information in addition to a medical image. As used herein, the age information is information that indicates the age of a classification target in the input medical image and, specifically, is information that indicates to which age group among the lower age group, the middle age group, and the higher age group a patient who has the lesion belongs. In Embodiment 1, the age information is known in advance from information such as medical records and is associated in advance with each of the plurality of medical images that may serve as a classification target.

The classification processor 115, using the classifier 30 to which a cutoff value that is adjusted by category (by age group) by the cutoff adjuster 113 is applied, classifies benignity or malignancy of a lesion that serves as a classification target in the unknown medical image accepted by the image accepter 114. First, the classification processor 115 selects a cutoff value to be used by the benign-malignant determiner 32, based on the age information accepted by the image accepter 114, as illustrated in FIG. 2. When specifically described, the classification processor 115 selects a cutoff value for an age group corresponding to the input age information from the cutoff values each of which is individually adjusted for one the plurality of age groups by the cutoff adjuster 113, as a cutoff value to be used by the benign-malignant determiner 32.

Next, the classification processor 115 inputs the unknown medical image accepted by the image accepter 114 to the classifier 30. In the classifier 30, the NN 31 calculates an estimated value of malignancy M of a lesion imaged in the input unknown medical image, using calculation parameters on which machine learning is performed by the trainer 111. The benign-malignant determiner 32 determines whether the lesion is benign or malignant by comparing the estimated value of the malignancy M calculated by the NN 31 with the cutoff value selected according to the age information. When specifically described, the benign-malignant determiner 32 determines the lesion to be malignant when the estimated value of the malignancy M exceeds the cutoff value, and determines the lesion to be benign when the estimated value of the malignancy M falls below the cutoff value. The classifier 30 outputs output information indicating benignity or malignancy determined by the benign-malignant determiner 32. The classification processor 115 classifies whether the lesion is benign or malignant for the unknown medical image, based on the output information output from the classifier 30, in this way.

The result outputter 116 outputs a result of classification performed by the classification processor 115. When specifically described, the result outputter 116 displays on the display 14 output information indicating whether the lesion in the unknown medical image is benign or malignant that is a result of classification performed by the classification processor 115. Alternatively, the result outputter 116 may output the output information by voice, or may output the output information to an external device via the communicator 15. Because of the output of information, the user can confirm a result of classification performed by the classification device 100.

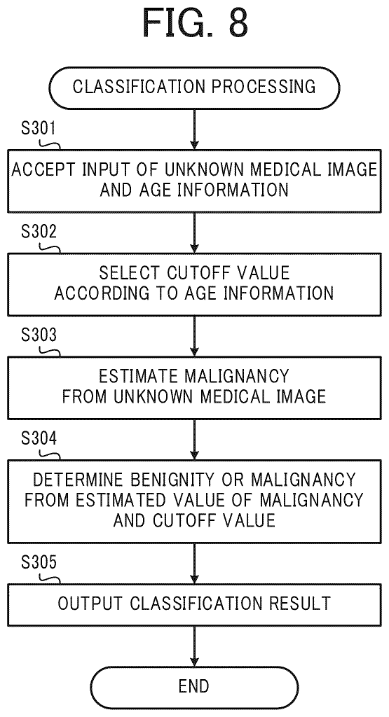

Next, with reference to FIG. 8, a flow of classification processing executed by the classification device 100 in the inference phase is described. The classification processing illustrated in FIG. 8 starts when the operation accepter 13 accepts a start instruction from the user while the classifier 30 generated by the classifier generation processing illustrated in FIG. 6 is stored in the storage 12.

When the classification processing is started, the processor 11 functions as the image accepter 114 and accepts input of an unknown medical image in which an unknown lesion serving as a classification target is imaged and age information (step S301). The processor 11 functions as the classification processor 115 and selects a cutoff value to be used by the benign-malignant determiner 32 according to the input age information (step S302). When specifically described, the processor 11 selects a cutoff value for an age group corresponding to the input age information from the cutoff values each of which is individually adjusted for one the plurality of age groups, as a cutoff value to be used by the benign-malignant determiner 32.

Next, the processor 11 functions as the classification processor 115 and inputs the unknown medical image to the classifier 30. In the classifier 30, the NN 31 estimates malignancy M of a lesion imaged in the input unknown medical image (step S303). The benign-malignant determiner 32 determines whether the lesion is benign or malignant by comparing the estimated value of the malignancy M calculated by the NN 31 with the cutoff value selected according to the age information in step S302 (step S304). The processor 11 acquires such a result of classification of benignity or malignancy performed by the classifier 30. Next, the processor 11 functions as the result outputter 116 and outputs output information indicating the acquired classification result (step S305). Consequently, the classification processing illustrated in FIG. 8 terminates. In the classification processing as described above, since the classifier 30 with the cutoff values adjusted by age group in the classifier generation processing is used, it is possible to accurately classify benignity or malignancy of a lesion even when there is a bias in the training data 121 with respect to the age groups.

As described in the foregoing, the classification device 100 according to Embodiment 1 sets, as target values for sensitivity and specificity that serve as classification indices of the classifier 30, target values common to a plurality of age groups, and adjusts the cutoff values by age group in such a way that the sensitivity and specificity evaluated using the evaluation data 122 divided by age group come close to the target values. The classification device 100 according to Embodiment 1, using the classifier 30 with the cutoff values adjusted by age group, classifies whether an unknown lesion imaged in an unknown medical image is benign or malignant. As described above, since the classification device 100 according to Embodiment 1 adjusts the cutoff values of the classifier 30 by age group, even when training images are not collected in a balanced manner for each of a plurality of age groups and there is a bias in the training data 121 with respect to the age groups of patients, it is possible to set an appropriate balance between sensitivity and specificity across all age groups. As a result, a stable classification result can be obtained.

In particular, at clinical sites, many cases at a young age are benign diseases. Therefore, collection of a sufficient number of training images of malignant cases at a young age involves difficulty. When an imbalance in the distribution of diseases in the training data 121 occurs in this way, bias is likely to occur in the classification result. For example, when the number of malignant cases is smaller than the number of benign cases, a classification result is likely to be biased toward the benign side. In contrast, the classification device 100 according to Embodiment 1 individually sets a cutoff value for each of a plurality of age groups and dynamically sets the cutoff value according to the age group of a lesion serving as a classification target. Therefore, even when there is a large bias in the case distribution of the training data 121, a stable classification result can be obtained.

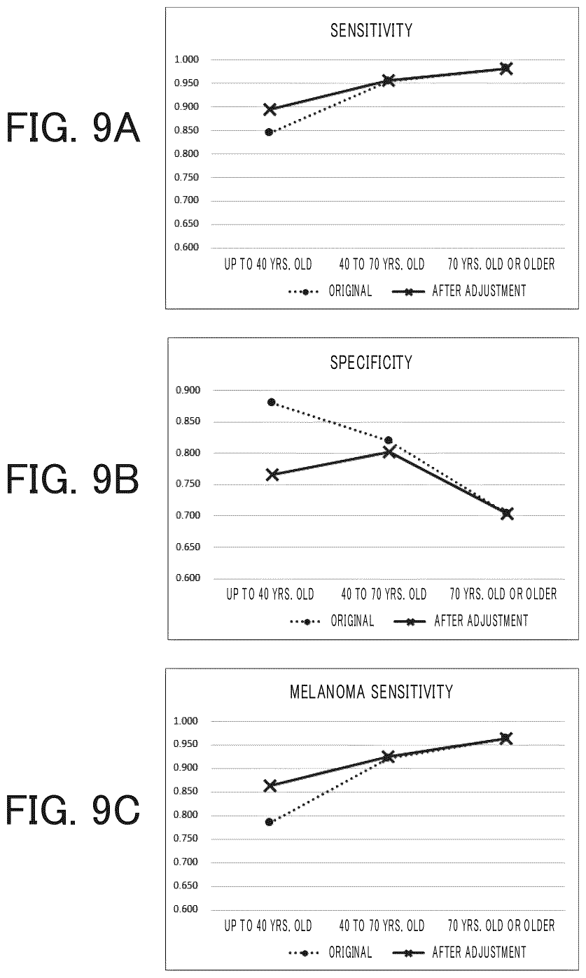

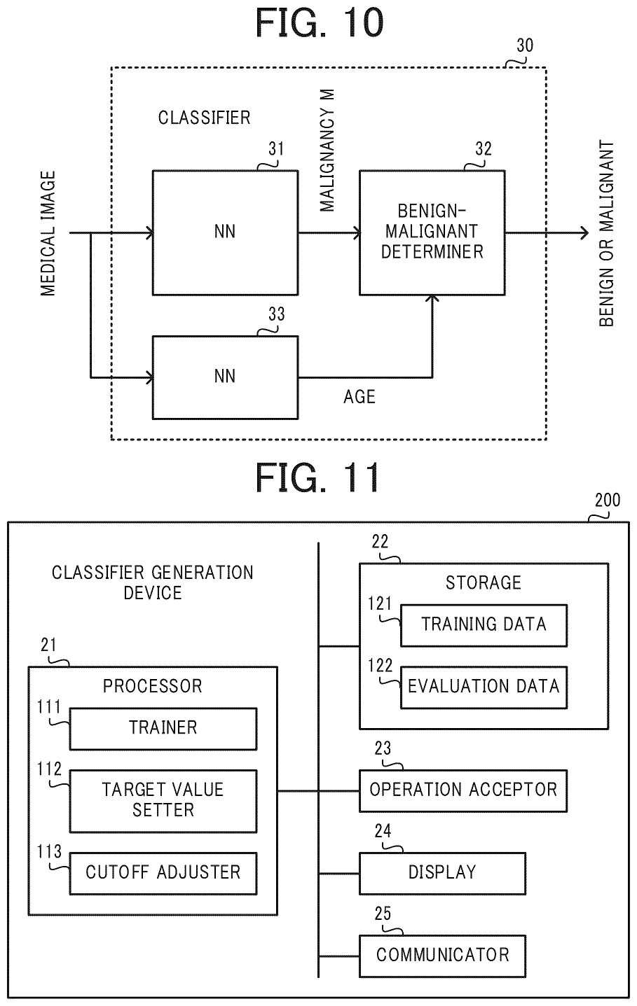

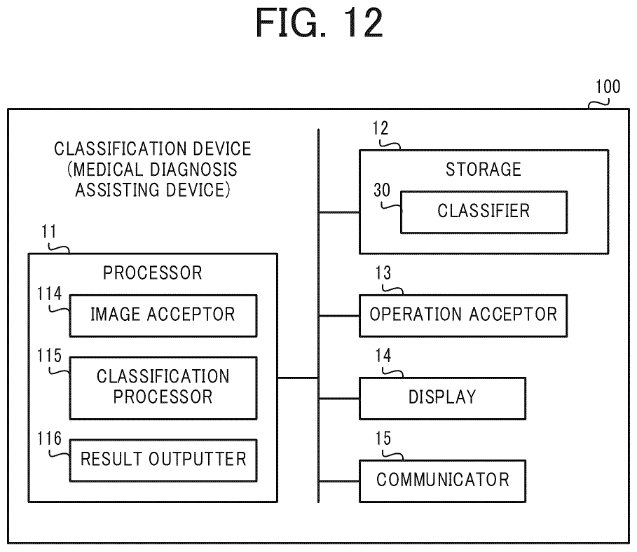

A result of evaluation in which the classifier 30 according to Embodiment 1 as described above is evaluated by experiment is illustrated in FIGS. 9A to 9C. In FIGS. 9A to 9C, the abscissa represents age groups of patients imaged in medical images by dividing the ages into three age groups, namely a lower age group (up to 40 years old), a middle age group (40 to less than 70 years old), and a higher age group (70 years old or older). The ordinates in FIGS. 9A and 9B represent sensitivity and specificity of the classifier 30 that are evaluated without limiting the type of lesion, respectively. In addition, the ordinate in FIG. 9C represents sensitivity of the classifier 30 that is evaluated only when a lesion is melanoma. As an evaluation method, using a plurality of evaluation images divided by age group, sensitivity or specificity of the classifier 30 that uses cutoff values adjusted by age group by a method described in Embodiment 1 and sensitivity and specificity of a conventional classifier that uses a cutoff value common to the plurality of age groups are evaluated by age group. In FIGS. 9A to 9C, the solid line indicates results of evaluation using the classifier 30 in Embodiment 1, and the dashed line indicates results of evaluation using the conventional (original) classifier.

As a result, as illustrated in FIGS. 9A to 9C, although no significant difference between the sensitivity and specificity of the classifier 30 of Embodiment 1 and the sensitivity and specificity of the conventional classifier was observed in the middle age group and the higher age group, differences were observed in the lower age group. More specifically, although the sensitivity and specificity in the lower age group were largely different from the sensitivity and specificity in other age groups when the conventional (original) classifier was used, the sensitivity and specificity in the lower age group come close to the sensitivity and specificity in other age groups when the classifier 30 of Embodiment 1 was used. In particular, in the result for the melanoma illustrated in FIG. 9C, sensitivity in the lower age group was largely improved when the classifier 30 of Embodiment 1 was used, compared with the case where the conventional classifier was used. Since melanoma is a dangerous malignant tumor, it is particularly important to classify the melanoma with high accuracy. Meanwhile, while melanoma is rare and there are few cases regardless of age, there are particularly few cases of melanoma at a young age. Therefore, collecting melanoma cases at a young age as the training data 121 involves difficulty. In contrast, it has been confirmed that when the classifier 30 in Embodiment 1 is used, melanoma in the lower age group, where there are fewer cases, can be classified with sensitivity close to sensitivity in the other age groups. By the experimental result described above, it is evident that the classifier 30, which uses cutoff values adjusted by age group, is effective in obtaining stable classification results even in the case where there is a bias in the case distribution in the training data 121.

Next, Embodiment 2 is described. Descriptions of the same constituent components and functions as those in Embodiment 1 are omitted. In Embodiment 1, the image accepter 114 accepts, in addition to input of an unknown medical image, input of age information of a classification target in the medical image. In contrast, in Embodiment 2, an image accepter 114 does not accept input of age information, and a classifier 30 estimates age of a classification target from a medical image.

Specifically, as illustrated in FIG. 10, the classifier 30 includes an NN 33, in addition to an NN 31 and a benign-malignant determiner 32 described in Embodiment 1. The NN 33 is an estimation unit that estimates the age of a classification target imaged in a medical image for input of the medical image. In other words, the NN 33 functions as a category estimator that estimates a category to which the classification target in the input medical image belongs from a plurality of categories. The NN 33 is, as with the NN 31, constructed by a neural network with a multi-layer structure, as an example. While the NN 31 calculates and outputs an estimated value of malignancy M of a classification target imaged in a medical image for input of the medical image, the NN 33 calculates and outputs an estimated value of the age of the classification target imaged in the medical image for the input of the medical image. The NN 33 is trained by machine learning in a training phase, as with the NN 31. When specifically described, a trainer 111 inputs a plurality of training images to each of which a correct age LA is attached as a correct label to the NN 33, and trains the NN 33 to learn calculation parameters in such a way that estimated value of age output from the NN 33 comes close to the correct age LA attached to the input training image.

In an inference phase, when an unknown medical image is input by the image accepter 114, a classification processor 115 inputs the unknown medical image to the classifier 30. In the classifier 30, the NN 31 calculates an estimated value of the malignancy M of a lesion imaged in the input unknown medical image. The benign-malignant determiner 32 selects a cutoff value corresponding to the age estimated by the NN 33 from cutoff values adjusted by age group by a cutoff adjuster 113. The benign-malignant determiner 32 determines whether the lesion is benign or malignant by comparing the estimated value of the malignancy M calculated by the NN 31 with the selected cutoff value. The classifier 30 outputs output information indicating benignity or malignancy determined by the benign-malignant determiner 32. The classification processor 115 classifies whether the lesion is benign or malignant for the unknown medical image, based on the output information output from the classifier 30, in this way. As described above, since the classifier 30 according to Embodiment 2 can estimate the age of a classification target from a medical image, the classifier 30 is capable of classifying benignity or malignancy of a classification target, using one of the cutoff values adjusted by age group even for a medical image where the age of the classification target is not known.

Next, Embodiment 3 is described. Description of the same constituent components and functions as those in Embodiments 1 and 2 are omitted. The classification device 100 according to Embodiments 1 and 2 described above includes, as illustrated in FIG. 1, both functions in the training phase and functions in the inference phase. In contrast, a classification device 100 according to Embodiment 3 does not include functions in a training phase, and a classifier generation device 200 that is a separate device from the classification device 100 includes the functions in the training phase.

Specifically, as illustrated in FIG. 11, the classifier generation device 200 according to Embodiment 3 includes a processor 21, a storage 22, an operation accepter 23, a display 24, and a communicator 25. Since hardware configurations of the constituent components described above are the same as the processor 11, the storage 12, the operation accepter 13, the display 14, and the communicator 15 in the classification device 100, descriptions thereof are omitted. The processor 21 includes, as functions in the training phase, a trainer 111, a target value setter 112, and a cutoff adjuster 113. In the processor 21, a CPU functions as the above-described functional components by retrieving programs stored in a ROM into a RAM and executing the programs to perform control. In addition, the storage 22 stores training data 121 and evaluation data 122 as data to be used in the training phase.

The respective functions of the processor 21 are the same as those in the trainer 111, the target value setter 112, and the cutoff adjuster 113 included in the classification device 100 in Embodiment 1. When specifically described, the processor 21 executes classifier generation processing illustrated in FIG. 6 by the functions of the trainer 111, the target value setter 112, and the cutoff adjuster 113. Because of this configuration, the processor 21 generates a classifier 30 that classifies benignity or malignancy of a lesion, using cutoff values adjusted by category (by age group).

On the other hand, the classification device 100 according to Embodiment 3 has a configuration as illustrated in FIG. 12. In the classification device 100 according to Embodiment 3, although not including functions in the training phase, the processor 11 includes, as functions in the inference phase, an image accepter 114, a classification processor 115, and a result outputter 116. Functions of the constituent components described above are the same as those in Embodiment 1. The classification device 100 acquires the classifier 30 generated by the classifier generation device 200 from the classifier generation device 200 by means of, for example, communication via the communicator 15, and stores the acquired classifier 30 in the storage 12. The processor 11 executes, by the functions of the image accepter 114, the classification processor 115, and the result outputter 116, classification processing illustrated in FIG. 8, using the classifier 30 acquired from the classifier generation device 200. Through this processing, the processor 11 classifies whether a lesion imaged in a medical image is benign or malignant. As described above, in Embodiment 3, since the functions in the learning phase and the functions in the inference phase are performed by separate devices, it becomes possible to perform more flexible operation, such as utilizing the classifier 30 generated by the classifier generation device 200 in a plurality of classification devices 100.

In a conventional technology, there are some cases where, depending on a classification target, it is difficult to collect training data for machine learning in a balanced manner. In such a case, since bias occurs in the training data, bias is liable to occur in a classification result. According to the present disclosure, it is possible to obtain stable classification results even when there is a bias in the training data. Although the embodiments of the present disclosure are described above, the above-described embodiments are only examples, and the scope of application of the present disclosure is not limited to the embodiments. That is, various applications of the embodiments of the present disclosure are possible, and all embodiments are included in the scope of the present disclosure.

For example, in the above-described embodiments, the classification indices that are index values relating to classification by the classifier 30 are sensitivity and specificity. However, the classification indices may be an index value other than sensitivity or specificity, such as a correct diagnostic rate and a relevance ratio. In addition, without being limited to using a plurality of classification indices such as sensitivity and specificity as classification indices, the cutoff values may be adjusted by category, using only one classification index, such as using only one of sensitivity or specificity as a classification index. In addition, in the above-described embodiments, the target value setter 112 searches for combinations of sensitivity and specificity that satisfy a specific condition by changing the cutoff value to a plurality of values, and sets a sensitivity A and a specificity A that satisfy the specific condition as target values common to a plurality of categories. However, without being limited to performing such a search using cutoff values, the target value setter 112 may, for example, set a typical value of sensitivity or specificity used for clinical purposes as a target value without verification.

In the above-described embodiments, the target value setter 112 sets target values common to a plurality of categories as target values of sensitivity and specificity, and the cutoff adjuster 113 adjusts cutoff values by category in such way that the sensitivity and specificity for each age group come close to the sensitivity A and the specificity A, which are target values common to the plurality of categories, respectively. However, without being limited to adjusting the cutoff values using the same target value for a plurality of categories, the cutoff adjuster 113 may change the target value with respect to each category. In other words, as long as a value used in the adjustment of cutoff values is a value based on a target value that is set by the target value setter 112 and that is common to the plurality of categories, the cutoff adjuster 113 may adjust cutoff values, using the target value itself common to the plurality of categories, or may adjust cutoff values, using a value determined with the target value common to the plurality of categories as a reference. Because of this configuration, balance between sensitivity and specificity in a specific category can be changed from other categories.

For example, in consideration of significant social impact of contracting cancer at a young age, the target value for sensitivity in the lower age group, which is a specific category, may be set higher than the target values for sensitivity in other age groups. In this case, the cutoff adjuster 113, while setting the target values for sensitivity and specificity in age groups other than the lower age group to the sensitivity A and the specificity A, which are target values common to the plurality of age groups set by the target value setter 112, sets the target values for sensitivity and specificity in the lower age group to a sensitivity B higher than the sensitivity A and a specificity B lower than the specificity A, respectively. The cutoff adjuster 113 adjusts the cutoff value for the lower age group in such a way that the sensitivity and specificity when the classifier 30 classifies benignity or malignancy of a classification target in the lower age group, using the evaluation data 122 divided by age come close to the sensitivity B and the specificity B, respectively. In addition, the weight of the sensitivity term in the loss function E may be increased by setting the value of α in the above-described equation (1) to be greater than 0.5, as needed. As described above, since it is possible to set different target values for sensitivity or specificity in the lower age group, which is a specific category, the sensitivity and specificity of the classifier 30 can be adjusted more flexibly even when risk or social impact varies between categories.

In the above-described embodiments, the description is made using the age group as an example of the category when cutoff values are adjusted by category. However, the category, without being limited to the age group, may, for example, be information indicating size, a body region, a race, or the like of a classification target. As used herein, the size of a classification target is size of a lesion (typically, long diameter) serving as a classification target. In general, there are fewer malignant cases of small sizes, as well as fewer malignant cases at a young age. Therefore, by dividing the evaluation data 122 by the size of a lesion and adjusting cutoff values by size, it is possible to obtain stable classification results, as with the above-described embodiments, even when there is a bias in the training data 121 with respect to size. In addition, the body region of a classification target refers to a region of the body where a lesion serving as a classification target exists (for example, the facial region, the palmoplantar region, the mucosal region, or the like). The race of a classification target refers to a race of a patient who has a lesion serving as a classification target (such as the white race, the black race, and the yellow race). Since there are also some cases where collecting training images in a balanced manner involves difficulty, depending on a body region or a race, by dividing the evaluation data 122 by body region or by race and adjusting cutoff values by body region or by race, stable classification results can be obtained even when there is a bias in the training data 121. As described above, there is an advantageous effect that using information that tends to cause a bias in the training data 121 as a category enables stable classification results to be obtained.

In the above-described embodiments, the classification device 100 classifies benignity or malignancy of a lesion. However, the classification device 100, without being limited to the configuration, may be a device that classifies a referral recommendation of a classification target. As used herein, the referral recommendation means recommending a patient having a lesion serving as a classification target to be referred to another hospital. For example, it is conceivable to refer a patient to a large-scale hospital capable of performing more specialized examinations from a small clinic. While in the above-described embodiments, the correct malignancy LM indicating malignant (1) or benign (0) is attached to each training image as a teacher label, in a case where the classification device 100 classifies a referral recommendation, information indicating whether referral recommendation is required (1) or not required (0) is attached to each training image as a teacher label in place of the correct malignancy LM. The trainer 111 performs machine learning, using such training data 121. Because of this configuration, the trainer 111 generates a classifier 30 that outputs output information indicating whether or not a referral recommendation is required for a lesion imaged in a medical image for input of the medical image. Alternatively, by, while keeping using the correct malignancy LM as the teacher label, setting a cutoff value used by the benign-malignant determiner 32 higher (that is, to the higher sensitivity side) than a case of classifying benignity or malignancy of a lesion, the classification device 100 may be used as a referral recommendation classifier.

In the above-described embodiments, the classification device 100 is a medical diagnosis assisting device that classifies whether a lesion imaged in a medical image is benign or malignant. However, the classification device 100 is not limited to serving as a medical diagnosis assisting device. For example, the classification device 100 may be an inspection device that accepts input of an inspection image in which a construction, such as a building, a road, and a bridge, is imaged and that classifies, as quality of the construction, whether there occurs an abnormality in the construction, based on cracks, front surface shape, and the like of the construction imaged in the inspection image. In this case, the classification target is not equivalent to a lesion imaged in a medical image, but a construction imaged in an inspection image. The quality of the classification target is not equivalent to benignity or malignancy of the lesion, but presence or absence of an abnormality in the construction.

In addition, the classification device 100, without being limited to outputting binary information as described above, such as the quality of a lesion (benign or malignant), whether or not a referred diagnosis is required (the presence or absence of referred diagnosis), and the quality of a construction (presence or absence of abnormality), as a classification result, may output information exceeding binary values as a classification result. For example, the classification device 100 may, using a classifier 30 capable of performing eight disease classification, output a classification result indicating which of the eight diseases a lesion corresponds to. As used herein, the eight diseases refer to, as an example, eight major diseases, namely melanoma, basal cell carcinoma, other malignant diseases, pigmented nevus, seborrheic keratosis, dermatofibroma, hemangioma, and other benign diseases. In this case, the NN 31 outputs probability values each of which indicates a probability that a lesion imaged in an input medical image corresponds to each of the eight diseases, in place of the malignancy M in the above-described embodiments. For example, when the input medical image is a melanoma image, the NN 31 is trained by the trainer 111 in such a way that a probability value corresponding to melanoma comes close to 1 and probability values corresponding to the other seven diseases come close to 0. The benign-malignant determiner 32 determines that a disease with the highest probability value among the eight probability values output from the NN 31 is a disease corresponding to the lesion imaged in the input medical image. For example, when the probability value of melanoma is the highest, the benign-malignant determiner 32 determines that the lesion is melanoma. The classifier 30 outputs a determination result determined by the benign-malignant determiner 32 as described above, as output information. In the classification device 100, the classification processor 115 classifies which of the eight diseases the lesion imaged in the input medical image corresponds to, based on the output information output from the classifier 30, and the result outputter 116 outputs the classification result.

In the above-described embodiments, the processor 11 or 21 functions as respective constituent components illustrated in FIGS. 1, 11, or 12 by the CPU executing programs stored in the ROM or the storage 12. However, the processors 11 and 21 may be dedicated hardware. The dedicated hardware is, for example, a single circuit, a composite circuit, a programmed processor, an application specific integrated circuit (ASIC), a field-programmable gate array (FPGA), or a combination of the foregoing. When the processors 11 and 21 are dedicated hardware, each of the functions of the constituent components may be achieved by an individual piece of hardware, or the functions of the constituent components may be collectively achieved by a single piece of hardware. In addition, among the functions of the constituent components, some functions may be achieved by dedicated hardware and the other functions may be achieved by software or firmware. As described above, the processors 11 and 21 can achieve the above-described functions by hardware, software, firmware, or a combination of the foregoing.

By applying a program that defines the operation of the above-described classification device 100 or classifier generation device 200 to a computer, such as a personal computer and a cloud server, it is possible to cause the computer to function as the above-described classification device 100 or classifier generation device 200. In addition, a method for distributing such a program is arbitrarily determined, and the program may be distributed stored in a non-transitory computer-readable recording medium, such as a compact disk ROM (CD-ROM), a digital versatile disk (DVD), a magneto optical disk (MO), and a memory card, or may be distributed via a communication network, such as the Internet. In addition, the above-described classification device 100 or classifier generation device 200 may be a system including a server and a device.

The foregoing describes some example embodiments for explanatory purposes. Although the foregoing discussion has presented specific embodiments, persons skilled in the art will recognize that changes may be made in form and detail without departing from the broader spirit and scope of the invention. Accordingly, the specification and drawings are to be regarded in an illustrative rather than a restrictive sense. This detailed description, therefore, is not to be taken in a limiting sense, and the scope of the invention is defined only by the included claims, along with the full range of equivalents to which such claims are entitled.

1. A medical diagnosis assisting device,
wherein the medical diagnosis assisting device classifies a classification target in a medical image by (i) using a classifier in which cutoff values are adjusted by category in such a way that an index value that is evaluated using evaluation data divided by category comes close to a target value common to a plurality of categories and (ii) comparing an estimated value of the classification target with one of the cutoff values.

2. The medical diagnosis assisting device according to claim 1, wherein the index value is at least one of sensitivity of the classifier or specificity of the classifier, and the cutoff values are adjusted by category in such a way that at least one of the sensitivity or the specificity in a case where the classifier classifies the classification target by category, using the evaluation data comes close to the target value.

3. The medical diagnosis assisting device according to claim 2,
wherein as the target value, a target value for the sensitivity of the classifier and a target value for the specificity of the classifier are set, and
the target value for the sensitivity is set to a higher value than the target value for the specificity.

4. The medical diagnosis assisting device according to claim 2, wherein a cutoff value in a specific category among the plurality of categories is adjusted in such a way that the sensitivity and the specificity in a case where the classifier classifies the classification target in the specific category, using the evaluation data comes close to a value higher than a target value for the sensitivity and a value lower than a target value for the specificity, respectively.

5. The medical diagnosis assisting device according to claim 1, wherein in a case where the medical image is input, the classifier classifies the classification target in the input medical image by comparison between a cutoff value corresponding to a category to which the classification target belongs among the cutoff values adjusted by category and the estimated value.

6. The medical diagnosis assisting device according to claim 5,
wherein the classifier
estimates, in the case where the medical image is input, the category to which the classification target in the input medical image belongs from the plurality of categories, and
classifies the classification target by comparison between the cutoff value corresponding to the estimated category among the cutoff values adjusted by category and the estimated value.

7. The medical diagnosis assisting device according to claim 1, wherein the category is information that indicates at least one of age, size, a body region, or a race of the classification target.

8. The medical diagnosis assisting device according to claim 1, wherein the classifier classifies benignity or malignancy of the classification target by comparison between an estimated value of benignity or malignancy of the classification target in the medical image and one of the cutoff values.

9. A medical diagnosis assisting method,
wherein the medical diagnosis assisting method classifies a classification target in a medical image by (i) using a classifier in which cutoff values are adjusted by category in such a way that an index value that is evaluated using evaluation data divided by category comes close to a target value common to a plurality of categories and (ii) comparing an estimated value of the classification target with one of the cutoff values.

10. A system including a server and a device, the system comprising
one or more processors,
wherein the one or more processors classify a classification target in a medical image by (i) using a classifier in which cutoff values are adjusted by category in such a way that an index value that is evaluated using evaluation data divided by category comes close to a target value common to a plurality of categories and (ii) comparing an estimated value of the classification target with one of the cutoff values.
